(19) United States
(12) Patent Application Publication
(10) Pub. No.: US 2015/0297395 A1
(43) Pub. Date: Oct. 22, 2015

(54) COLD OR HOT FOMENTATION BAG FORMING STRUCTURE

(71) Applicant: TAIWAN STANCH CO., LTD., Taipei City (TW)

(72) Inventor: Chun-Sheng HUANG, Taipei City (TW)

(73) Assignee: TAIWAN STANCH CO., LTD., Taipei City (TW)

(21) Appl. No.: 14/257,453

(22) Filed: Apr. 21, 2014

(51) Int. Cl. A61F 7/02

(52) U.S. Cl. CPC A61F 7/02; A61F 2007/022

(57) ABSTRACT

The cold or hot fomentation bag forming structure includes a bag body having a bottom, an upper opening, a folded end formed on an upper edge and a folded portion adjacent to the folded end, wherein the folded portion has a predetermined length; a bag opening member disposed on the upper opening and connected to a cover opening or closing the bag opening member; a neck portion extending from the bag opening member; and at least one elastic gasket holding the neck portion and held by the folded portion; and at least one collar holding an outer surface of the elastic gasket and an outer surface of the folded portion.

### BACKGROUND OF THE INVENTION

1. Field of the Invention

The invention relates to a cold or hot fomentation bag forming structure, and more particularly to a cold or hot fomentation bag forming structure utilizing sutures rather than adhesives to improve deterioration caused by the adhesive and extend the service life.

2. Description of the Related Art

Human body has a normal temperature of 37° C. Illness or injure often causes body temperature rising. At this time, cold fomentation bags are often used for reduction of the body temperature. In addition, work fatigue or excessive exercise may cause muscle aches which can be eased by hot fomentation. According to experiment, the optimal temperature for hot fomentation is 40° C. to 50° C. When the hot fomentation is performed by hot towel, the temperature is rapidly decreased and lose the hot fomentation effect in a short period. However, the hot fomentation bag can keep the temperature for a longer time.

Referring to FIGS. 1 and 2, a conventional cold and hot fomentation bag includes a bag body 90 assembled with a bag opening member 91, and a cover is disposed on the bag opening member 91. A folded portion 92 is formed on an upper end of the bag 90, and an extending periphery 93 extends from the bag opening member 91. An adhesive layer 94 are formed on the extending periphery 93, and an inner surface of the folded portion 92 is bonded to the adhesive layer 94. A collar 95 holds the bag body 90 and the bag opening member 91 together.

As the bag body 90 contains hot or cold media material, such as ice or hot water, when the conventional cold and hot fomentation bag is used for a long time, the hot or cold media material may enter the adhesive layer 94 along the arrow 96 of FIG. 2 and damage the adhesive layer 94, which causes the bag body 90 separated from the bag opening member.

### BRIEF SUMMARY OF THE INVENTION

The invention provides a cold or hot fomentation bag forming structure. The cold or hot fomentation bag forming structure in accordance with an exemplary embodiment of the invention includes a bag body having a bottom, an upper opening, a folded end formed on an upper edge and a folded portion adjacent to the folded end, wherein the folded portion has a predetermined length; a bag opening member disposed on the upper opening and connected to a cover opening or closing the bag opening member; a neck portion extending from the bag opening member; and at least one elastic gasket holding the neck portion and held by the folded portion; and at least one collar holding an outer surface of the elastic gasket and an outer surface of the folded portion.

In another exemplary embodiment, the folded portion is formed as a loop shape adapted to the neck portion.

In yet another exemplary embodiment, the folded portion of the loop shape is formed by suture.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 is a partially exploded view of a conventional fomentation bag; and

FIG. 2 is cross sectional enlarged view of FIG. 1;

FIG. 3 is a perspective view of a cold or hot fomentation bag of the invention, wherein the cover is separated;

FIG. 4 is a exploded perspective view of a cold or hot fomentation bag of the invention with cover removed;

FIG. 5 is a perspective view of a cold or hot fomentation bag of the invention;

FIG. 6 is a cross sectional view of a cold or hot fomentation bag of the invention;

FIG. 7 is an enlarged view of the portion A of FIG. 6; and

FIG. 8 shows a collar added to the structure shown in FIG. 7.

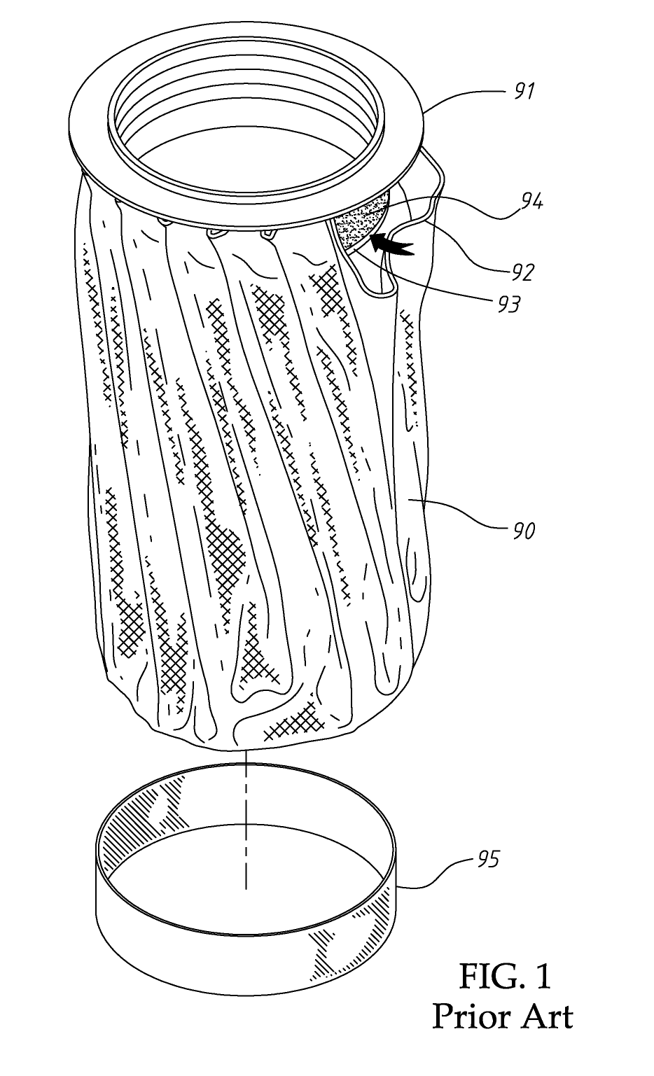

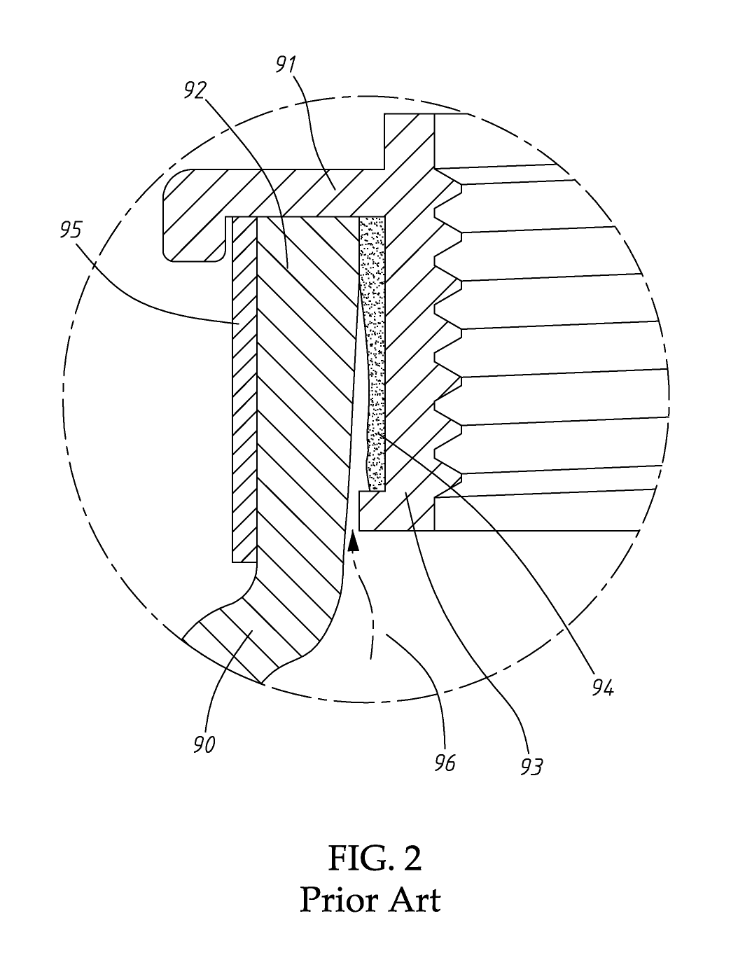

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

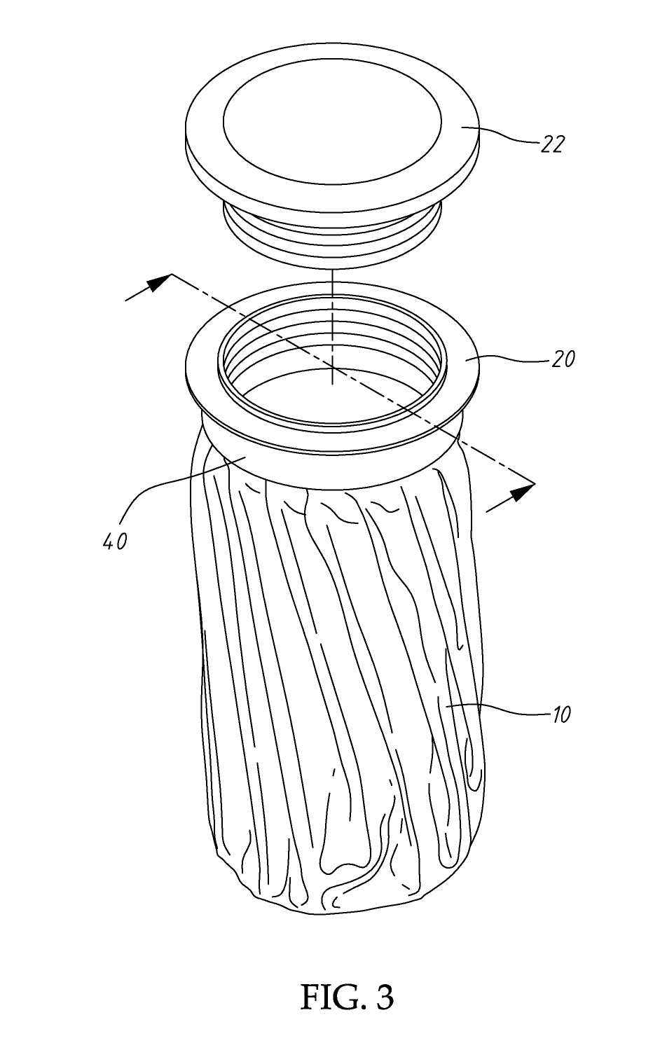

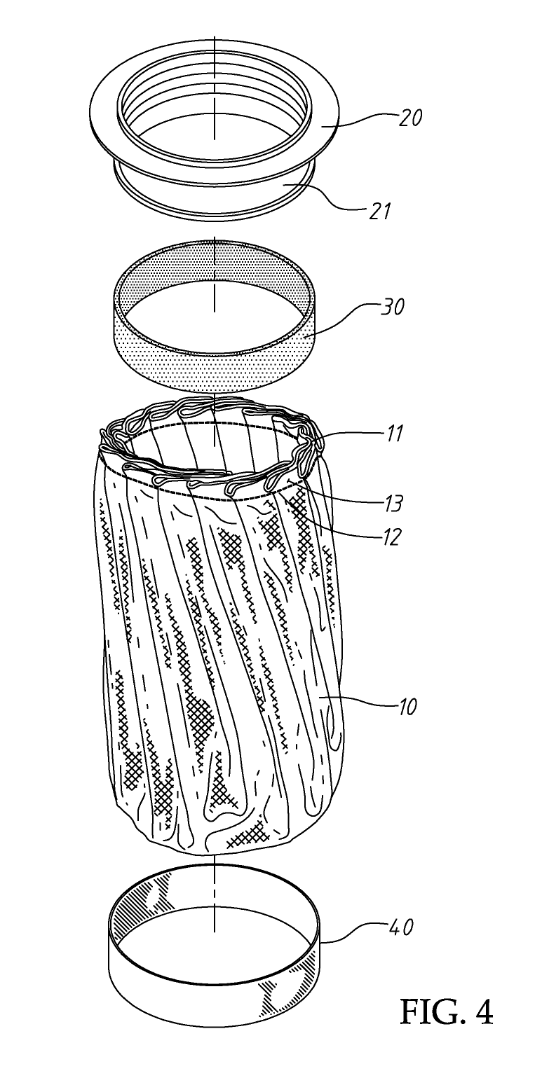

Referring to FIGS. 3 and 4, the cold or hot fomentation bag of the invention includes a bag body 10 having a bottom and an opening, a bag opening member 20 and a cover 22 opening or closing the bag opening member 20. The bag opening member 20 extends downwards to form a neck portion 21.

The bag body 10 is formed by folding bag material and has a folded end 11. In this embodiment, a suture 12 is sewed on a predetermined position below the folded end 11 to form a folded portion 13 having a ring shape adapted to the width of the neck portion 21 and holding the neck portion 21.

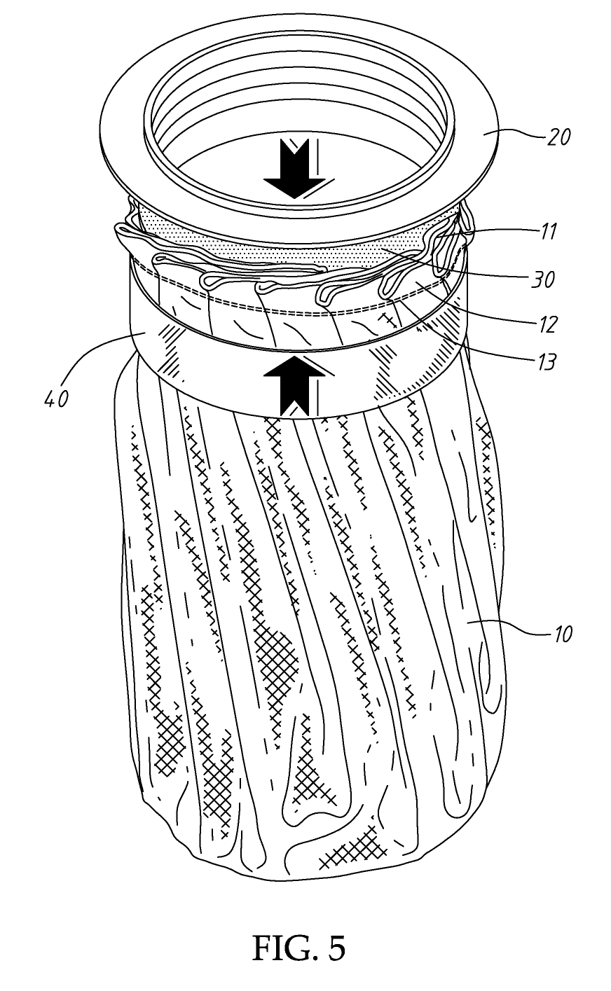

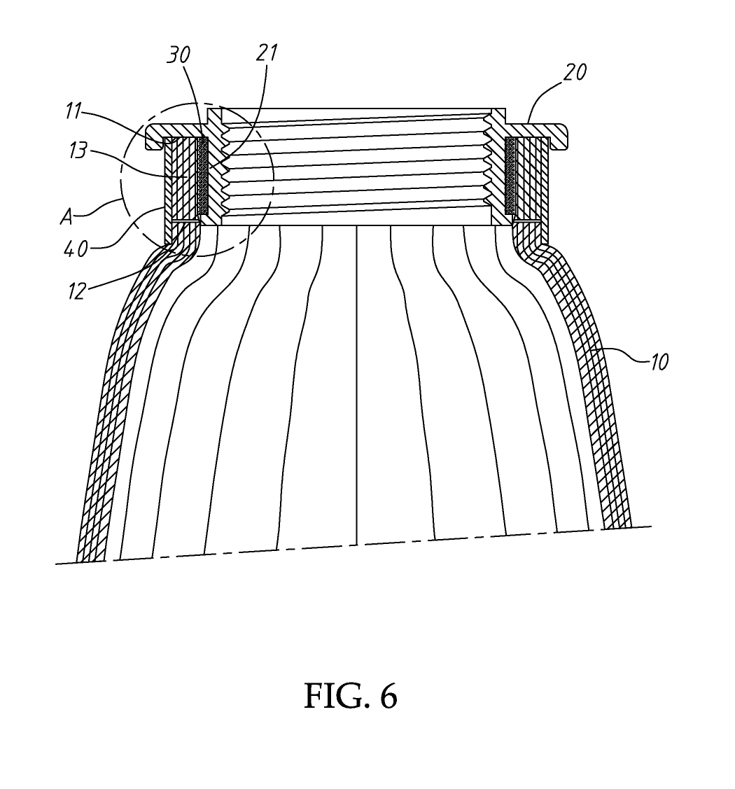

Referring to FIGS. 3 to 5, an elastic gasket 30 made of soft elastic material such as silicone can hold the neck portion 21 of the bag opening member 20, and the ring shaped folded portion 13 holds an outer surface of the elastic gasket 30. A collar 40 is disposed on the outer surface of the elastic gasket 30 and an outer surface of the folded portion 13 to hold the bag body 10 and the bag opening member 20 together (as shown in FIG. 6).

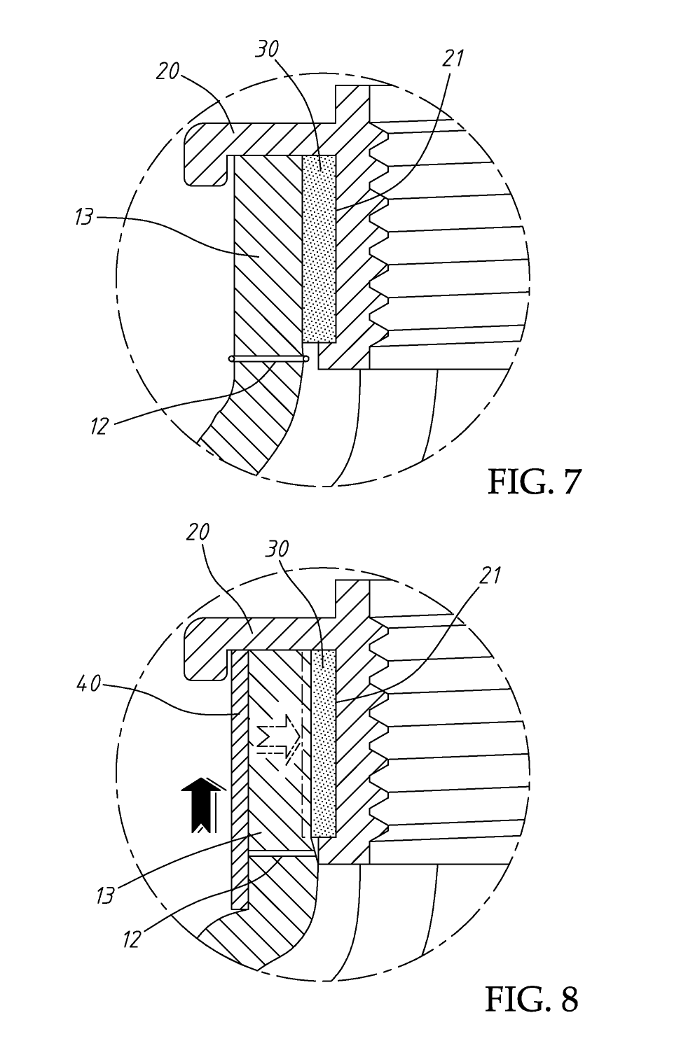

Referring to FIG. 7, when the folded portion 13 holds the elastic gasket 30, the elastic gasket 30 is not pressed. When the collar 40 holds the elastic gasket 30 and the folded portion 13, as shown in FIG. 8, the collar 40 press the folded portion 13 so as to press the elastic gasket 30 such that bag body 10 is sealed by the bag opening member 20.

Since the elastic gasket 30 is disposed between the bag body 10 and the bag opening member 20 for sealing, even the cold and hot media material seeps the elastic gasket 30, the elastic gasket 30 is not influenced by the cold and hot media material. The folded portion 13 which is formed by sutures form the holding structure without adhesive can improve the damage caused by the adhesive.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A cold or hot fomentation bag forming structure, comprising:
a bag body having a bottom, an upper opening, a folded end formed on an upper edge and a folded portion adjacent to the folded end, wherein the folded portion has a predetermined length;
a bag opening member disposed on the upper opening and connected to a cover opening or closing the bag opening member;
a neck portion extending from the bag opening member; and
at least one elastic gasket holding the neck portion and held by the folded portion; and
at least one collar holding an outer surface of the elastic gasket and an outer surface of the folded portion.

2. The cold or hot fomentation bag forming structure as claimed in claim 1, wherein the folded portion is formed as a loop shape adapted to the neck portion.

3. The cold or hot fomentation bag forming structure as claimed in claim 2, wherein the folded portion of the loop shape is formed by suture.
